(19) United States
(12) Patent Application Publication
(10) Pub. No.: US 2004/0024055 A1
(43) Pub. Date: Feb. 5, 2004

(54) Pharmaceutical composition containing the exract of saururus chinensis baill useful as an anticancer agent and a process for the preparation thereof

(76) Inventors: Jong- Cheon Hahm, Daejeon Metropolitan City (KR); Dae-Sang Lee, Daejeon Metropolitan City (KR); Jae-Pil Ko, Daejeon Metropolitan City (KR); In-Kyoung Lee, Seongnam-City (KR); Hyun-Woo Lee, ChungJu-City (KR); Jeong-Sook Park, Cheongju-City (KR)

Correspondence Address: DUANE MORRIS LLP, 100 COLLEGE ROAD WEST, SUITE 100, PRINCETON, NJ, 08540-6604, US

(21) Appl. No.: 10/276,599

(22) PCT Filed: May 17, 2001
(86) PCT No.: PCT/KR01/00818

(30) Foreign Application Priority Data
May 18, 2000 (KR) 2000-26801

(51) Int. Cl. C07D 307/02; A61K 31/34

(52) U.S. Cl. 514/461; 514/471; 549/491; 549/502

(57) ABSTRACT

The present invention generally relates to novel compounds HNP-98701A (epi-manassantin A), HNP-98701B and HNP-98701C (manassantin A), or mixture thereof (HNP-98701) as carcinostatis substance, preparation method thereof and carcinostatis pharmaceutical composition containing them as effective constituents and, more specifically, to novel compounds HNP-98701A and HNP-98701B, known compounds HNP-98701C, or mixture thereof and derivatives thereof obtained by extracting saururus with methanol, fractionating this with various organic solvents, separating ethyl acetate fraction with highest anticancer activity through thin-layer chromatography or column chromatography and purifying it, preparation method thereof and carcinostatis pharmaceutical composition containing them as effective constituents. The carcinostatis substances HNP-98701A, HNP-98701B and HNP-98701C of the present invention selectively affect cancer cell line cells and cause apoptosis-type cell death. Therefore, it can be used for development of selective carcinostatis substance, which has very superior anticancer activity and fewer adverse effects against normal cells compared with the conventional carcinostatis substances.

### FIELD OF INVENTION

The present invention generally relates to novel compounds HNP-98701A (E/E epi-manassantin A), HNP-98701B and HNP-98701C (manassantin A), or mixture thereof (HNP-98701) as carcinostatis substance, preparation method thereof and carcinostatis pharmaceutical composition containing them as effective constituents and, more specifically, to novel compounds HNP-98701A and HNP-98701B, known compounds HNP-98701C, or mixture thereof and derivatives thereof obtained by extracting saururus with methanol, fractionating this with various organic solvents, separating ethyl acetate fraction with highest anticancer activity through thin-layer chromatography or column chromatography and purifying it, preparation method thereof and carcinostatis pharmaceutical composition containing them as effective constituents.

### BACKGROUND OF INVENTION

About 10% of Southeast Asians are hepatitis carriers, and hepatitis is thought of as direct cause of liver cirrhosis and liver cancer. Statistically, it is reported that more than 95% of liver cancer was caused by hepatitis. Especially, Koreans' incidence rate and death rate related with liver cancer is the highest in the world. According to the statistics of 1991, Koreans' liver cancer incidence rate was 24.1 people per 100,000 populations, and according to the 1996 data of the Ministry of Health and Welfare, liver cancer incidence rate was 22.4%, which is only next to 40.7% of stomach cancer. Also, according to the statistics of 1995 of Korea, death rate caused by liver cancer was 22.0 people per 100,000 populations, which is just next to that of the stomach cancer—26.5 people per 100,000 populations. This is the highest in the world for liver cancer death rate.

With this high liver cancer incidence rate and liver cancer death rate at the background, research, development and commercialization of hepatitis vaccine, hepatitis treatment and liver cancer treatment are actively carried out. However, various treatments developed until now have many problems. For example, the currently marketed hepatitis vaccine has insufficient antigenicity to be effective as vaccine, and most hepatitis treatments are focused on improving liver functions rather than directly affecting hepatitis virus. Recently, a hepatitis treatment that inhibits propagation of hepatitis virus was developed. However, it cannot be applied to all hepatitis patients. Therefore, hepatitis patients are exposed to the possibility of developing to liver cancer.

For currently used liver cancer treatments, there are 5-fluorouracil (5-FU), sytarabine and alkyloxane. These compounds are focusing to inhibit growth of cancer cell rather than destroying it. So, they are not ultimate treatment for liver cancer. “Holium-chitosan complex treatment” (DW-166HC), which is known as epoch-making liver cancer treatment, is not yet proved for its stability and effect. Therefore, a long-term clinical trial against many patients is required. And if more than two tumor lumps are spread over several organs or spread to other organs, if there is abdominal dropsy or jaundice, or if several blood vessels are connected to the tumor lump, this method cannot be applied. Moreover, this method can be applied only through doctor's operation in hospital.

Because liver is involved with all metabolisms, development of liver cancer treatment is a very difficult task. However, if a selectively active substance that shows maximum toxicity against liver cancer cell and minimum toxicity against normal cell is developed, it can be very useful for epochal liver cancer treatment.

On the other hand, in the West, prostate cancer is a male disease whose incident rate is as high as that of breast cancer in women. It is one of the most frequently diagnosed male diseases in the US. Each year, more than 300,000 patients are diagnosed as prostate cancer, and about 41,400 patients die of prostate cancer every year. In the US, prostate cancer is the second to lung cancer as cause of death.

Recently, about 40 per 100,000 elders older than 60 years are attacked by prostate cancer. Especially, most of prostate cancer patients are reported to have had prostatic hypertrophy symptom at early stage. This arouses attention of prostatic hypertrophy patients, which amounts to more than 60% of elders older than 60. However, the biggest problem in prostate cancer treatment is that most patients are reluctant to receive prostate cancer treatment due to the adverse effect of impotence. Therefore, research on prostate cancer has been ignored relatively. 10 years ago, the only treatment of prostate cancer was operation, which causes adverse effects like impotence and incontinence. However, many epoch-making prostate cancer treatments are being developed. For example, there are immune system stimulating method of Cell Genesys, antisense technology of Isis Pharmaceuticals, Inc. and Novartis AG, adenoviral-p53 gene therapy of Introgen Therapeutics, Inc., radioactive therapy of Theragenics, CaverMap Surgical Aid of UroMed Corp. and Proscar of Merck Co., a prostatic hypertrophy treatment. However, these researches are only at early stages.

As mentioned above, although prostate cancer is the most frequent cancer in male genital organ, many patients are reluctant to receive treatment because of the adverse effect of impotence and its complete cure is not easy. Therefore, if a selectively active substance that shows maximum toxicity against prostate cancer cell and minimum toxicity against normal cell is developed from natural substance, it will be very useful for treatment of prostate cancer treatment and it will contribute to human health.

Saururus is a plant that belongs to the family Saururaceae, the order Piperales. The family Saururaceae plants comprise 5 genuses and 7 species, and are distributed mainly in North America and Asia. For the genuses Saururaceae that have a worldwide distribution, there are saururus (Saururus chinensis Baill) and western saururus (Saururus cernuus). For the family Saururaceae plants that grow wild in Korea, saururus and houttuynia (Houttuynia cordata Thunb; fishy herb) are reported. All of these are perennial plants that grow in marshy area. Although they are sold without discrimination in general herbal medicine market, saururus and houttuynia are undeniably different plants. Saururus grows wildly mainly in Jeju Island. It is specified as endangered species and is actively being rehabilitated. It is being grown in large unit in Geochang of Gyeongsangnam-do and trial culturing is being performed by ChungcheongNam-Do Agricultural Research and Extension Services.

Saururus is a perennial plant with white crosswise rhizome and fibrous root. It is about 30-90 cm tall, and it has upright hairless stem. Its lower part is slanted. It has alternate leaves and ovate or ovally lanceolate petioles. Its leaf is about 5-14 cm long and 4-6 cm wide, and has five-forked vein. Early in summer, 2-3 leaves at the upper part of the stem tend to turn white. Its flower is white and hermaphroditic. It is 10-15 cm tall and blooms between June and August. It has 6-7 stamens and one pistil. Its ovary has 3-5 carpets. Saururus is also called as “three-white herb” because three leaves at the top stem turn white during blooming season or because its root, leaf and flower are white.

Saururus has been used as folk medicine in China from a long time ago. According to Jungyak Dictionary, saururus is said to be effective in dropsy, beriberi, jaundice, leucorrhoea and tumor. Therefore, it is effective in liver diseases.

According to Tang Herbal, saururus is said to be effective in dropsy, beriberi, smooth defecation and urination, clearance of phlegm and liver hardening. Therefore, it seems to be effective in liver cirrhosis and liver cancer. Also, the effectiveness in smooth defecation and urination shows that it is effective in prostatic hypertrophy and prostate cancer.

According to Chinese Pharmaceutical Dictionary, saururus is said to be effective in dropsy, beriberi, smooth defecation and urination, clearance of phlegm and liver hardening, and according to Chinese Medicinal Dictionary, saururus is said to be effective in phlegm, liver hardening, smooth defecation and urination, malaria, hot lower chest, dropsy and beriberi.

The effectiveness of saururus is also written in Herbal Seubyu, Illustrated Plant Guide, Yeongnam Herb-Collecting, Gwangseo Central Pharmaceutical, Herbal Chujin and Honam Pharmaceutical. And, according to Thousand Prescriptions for Carcinostatis Chinese Medicine, saururus is said to be effective in liver cancer.

Saururus has long been used as folk medicine in Korea, too. According to Pull-color Natural Drug Dictionary, saururus is said to be effective in smooth defecation and urination, dropsy, beriberi, hepatitis, jaundice and tumor. Therefore, it is effective in liver diseases, defecation/urination and cancer.

In the US, Chaubal, et al. reported that many sesquiterpene compounds are included in western saururus (Saururus cernuus) [Chaubal et al., Phytochemistry, 14: 595-596, 1975], and Rao et al. separated new neolignan active substances SC-8 and SC-9 and found that these substances have neuroleptic activity [Rao et al., Dissertation Abstract International, 45(6): 2384B, 1981]. Afterward, Rao et al. continued their research and separated manassantin A, manassantin B and saucerneol from saururus and elucidated their structures. Among these, manassantin A was shown to have strong neuroleptic activity [Rao et al., Tetrahedron Lett. 24(45): 4947-4950, 1983]. Based on these results, Rao et al. performed mouse toxicity test to find out central nerve depression effect of manassantin A. When manassantin A was administered into the abdominal cavity of mouse with 0.21±0.02 mg/kg of IC₅₀, spontaneous movement was reduced and depression of amphetamine-induced stereotypy was observed. On the other hand, administration of manassantin A to the level of LD₅₀ did not cause stiffness or ptosis as in haloperidol. Therefore, manassantin A has selective neuroleptic activity [Rao et al., Pharmacol. Res. Comm. 19(9): 629-639, 1987]. Also, Rao et al. separated lactam compounds aristololactam BII (cepharanone) and sauristolactam from western saururus [Rao et al., J. Nat. Prod., 53(2): 309-312, 1990].

In China, Xu et al. reported saururus analysis method through coulometric titration of flavonoid and hyperin [Xu et al., Acta Pharmaceutica Sinica, 21(4): 306-309, 1986; [symbol] [symbol] , 8(4): 223-225, 1988]. Wang et al. found that chloroform fraction extracted from saururus has strong antihypertensive activity, and separated sauchinone lignan, physcion, emodin and physion-8-D-glucopyranose anthraquinones and cepharanone B alkaloid from saururus [Wang et al., Heterocycles, 43(5): 969-975, 1996].

In Japan, while there is almost no research on saururus, research on houttuynia is actively carried out.

As shown above, there is no research result in foreign countries including US, China and Japan that saururus has anticancer activity. And, there is no report that effective carcinostatis component was separated from saururus.

In Korea, Kwak reported that aqueous extract, methanol extract, butanol fraction extract and quercetin of saururus are effective in pain killing, removal of fever and inflammation [Kyung Hee University Doctoral Thesis, 1988]. In particular, Kwak reported the relaxation effect of saururus extract on extracted and inverted internal canal of mouse and guinea pig, contracted by acetylcholine, barium and histamine. The result seems to agree with the research of Rao et al. Besides, Kwak reported that some of the fractions have antihistamine, blood pressure drop and antibacterial effect. Choi extracted volatile component, fatty acid, amino acid and flavonoid from saururus and reported that aqueous extract of saururus has antibacterial activity [Kyung Hee University Doctoral Thesis, 1989]. Lee separated presumable terpenoids from saururus, but he reported that this component does not have physiological activity [Seoul National University Masters Thesis, 1988]. Kwon separated a substance that has liver cell protection activity from saururus. He elucidated the structure of the substance and reported that it is a quercetin glycoside of flavonoid [Seoul National University Masters Thesis, 1994].

Besides Choi and Chung reported research on flavonoid component of saururus [Analytical Science & Technology, 4(3): 285-288, 1991; Analytical Science & Technology, 7(1): 11-15, 1994; J. of Basic Sciences, Cheju Nat. Univ. 8(1): 137-142, 1995], research on essential oil component [Analytical Science & Technology, 2(2): 259-262, 1988; Kyung Hee University Theses 18: 341-347, 1989; Cheju Nat. Univ. Journal 33: 105-111, 1991] and research on fatty acid and amino acid components [Analytical Science & Technology, 2(2): 285-292, 1989; Cheju Univ. Jour. (Natural Sci.), 35: 111-118, 1992].

As mentioned above researches on saururus in Korea are actively carried out in the basic field of component analysis. However, none of the more than 50 components of saururus is reported to have anticancer activity, yet.

Number of saururus-related patents is also small. Outside Korea, there is one in filed in the US (U.S. Pat. No. 4,619,943). This is about neuroleptic, insecticidal and nematocidal effect of manassantin A, which is derived from western saururus, and no anticancer effect is disclosed. Inside Korea, only one patent (Korean Patent Publication No. 96-31440) was filed. This is about carcinostatis substance composition of chemical derivative of sauristolactam, which is one of the saururus components. However, no anticancer activity was detected when we separated sauristolactam from saururus and crystallized it.

As explained above, although saururus has traditionally been used for neoplasm treatment like tumor carcinoma in liver and urinary organs, specific research on anticancer component of saururus has not been reported yet. Among more than 50 components that are separated from saururus, none has been identified as carcinostatis substance, and only quercetin glycoside of flavonoid is presumed to be related with anticancer activity.

We separated new carcinostatis substance from saururus, which selectively affects liver cancer cell and prostate cancer cell and does not affect normal cell, it can be very useful for epochal liver cancer treatment. This substance will be useful for liver cancer and prostate cancer treatment. Also, we found that this new lead compound separated from saururus selectively affectds liver cancer cell and prostate cancer cell, and prepared pharmaceutical composition containing it as effective constituent, which can be very useful for treatment of cancers including liver cancer and prostate cancer.

### SUMMARY OF INVENTION

It is, therefore, an object of the present invention to provide novel compounds HNP-98701A (epi-manassantin A) and HNP-98701B and known compound HNP-98701C (manassantin A) and derivative thereof from saururus, is which have anticancer activity.

It is another object of the present invention to provide a preparation method of novel compounds HNP-98701A and HNP-98701B and known compound HNP-98701C from saururus through extraction.

It is further another object of the present invention to provide a pharmaceutical composition which contains the novel compounds HNP-98701A and HNP-98701B, known compound HNP-98701C or mixture thereof (HNP-98701) as effective constituent.

In order to attain the above objects, according to an aspect of the present invention, there are provided novel compounds having anticancer activity and obtained from saururus, expressed by Formula 1, Formula 2 and Formula 3, their derivatives and pharmaceutically available salts thereof.

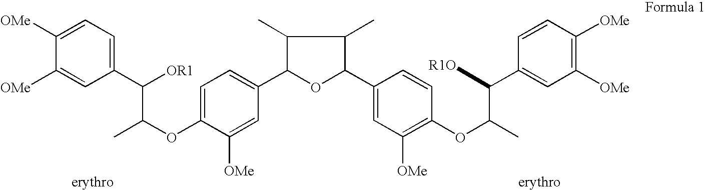

In Formula 1, R1 is selected from methyl, acetyl, alkyl, alkene, alkyne, amine, amide, cyano, thiocyano, aldehyde or halogen atom, and may be identical or different.

In Formula 1, the novel compound HNP-98701A, wherein R1 is H, is expressed by Formula 4. It also has anticancer activity and exists as epimer of manassantin A.

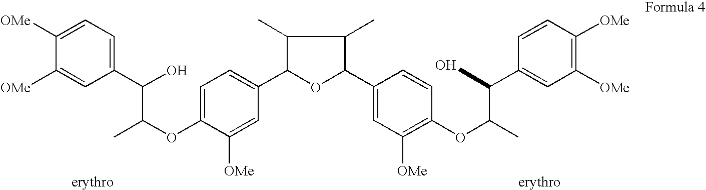

And, in Formula 1, acetyl derivative or methyl derivative with —COCH₃ or —CH₃ substituted R1 also has anticancer activity.

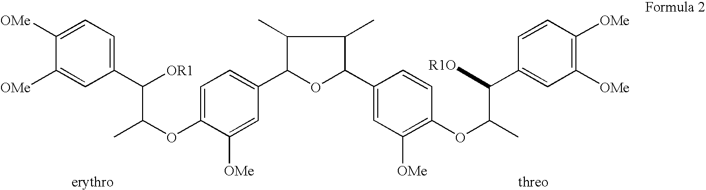

In Formula 2, R1 is selected from methyl, acetyl, alkyl, alkene, alkyne, amine, amide, cyano, thiocyano, aldehyde or halogen atom, and may be identical or different.

In Formula 2, the novel compound HNP-98701B, wherein R1 is H, is expressed by Formula 5. It also has anticancer activity and has the hybrid structure of manassantin A and epi-manassantin A.

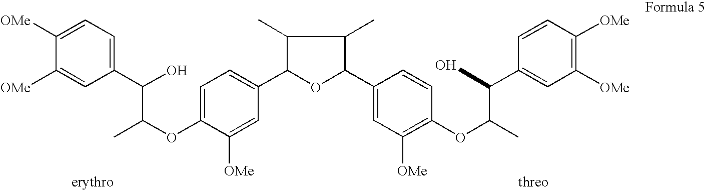

And, in Formula 2, acetyl derivative or methyl derivative with —COCH₃ or —CH₃ substituted R1 also has anticancer activity.

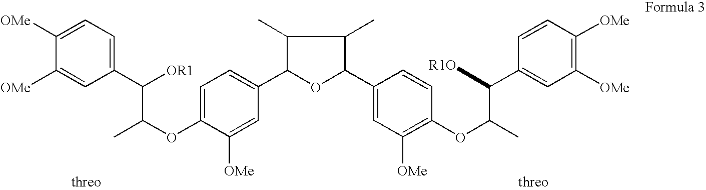

In Formula 3, R1 is selected from methyl, acetyl, alkyl, alkene, alkyne, amine, amide, cyano, thiocyano, aldehyde or halogen atom, and may be identical or different.

In Formula 3, the novel compound HNP-98701C, wherein R1 is H, is expressed by Formula 6. It is manassantin A with anticancer activity, and its epimer also has anticancer activity.

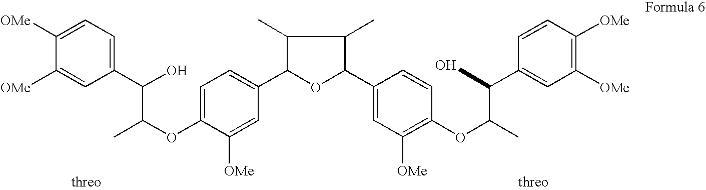

And, in Formula 3, acetyl derivative or methyl derivative with COCH₃ or —CH₃ substituted R1 also has anticancer activity.

And, mixture of the compounds expressed by Formulas 1, 2 and 3 also has anticancer activity.

The compounds expressed by Formulas 1, 2 and 3 can be used in the form of pharmaceutically available salt. Among these, the acid salt prepared using pharmaceutically available free acid is useful. The compounds expressed by Formulas 1, 2 and 3 can be prepared as pharmaceutically available acid salt by the conventional method. For free acid, organic acid and inorganic acid can be used. For inorganic acid, hydrochloric acid, bromic acid, sulfuric acid or phosphoric acid can be used. For organic acid, citric acid, acetic acid, lactic acid, tartaric acid, acetic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, galacturonic acid, embonic acid, glutamic acid or aspartic acid can be used.

Saururus was extracted with methanol and fractionated with various organic solvents. Among the organic solvent fractions, ethyl acetate extracted fraction that showed best anticancer activity was separated by thin-layer chromatography or column chromatography and purified by high performance liquid chromatography to obtain the novel compounds HNP-98701A (epi-manassantin A), HNP-98701B and HNP-98701C (manassantin A) or mixture (HNP-98701) thereof of the present invention. As can be seen in Formulas 4 and 6, HNP-98701A is an epimer of manassantin A, whose hydroxyl group (—OH) position is different from that of HNP-98701C. It was separated from saururus for the first time, and HNP-98701A and HNP-98701C have erythro-type epi-manassantin A structure and threo-type manassantin A structure, respectively.

Also, because HNP-98701B shows the sameNMR data as those of HNP-98701A and HNP-98701C, it can be concluded that it has symmetric structure, in which one hydroxyl group of manassantin A is threo-type and the other hydroxyl group is erythro-type, as in Formula 5.

In order to determine physical and chemical properties of HNP-98701A, HNP-98701B and HNP-98701C, general properties, solubility, UV spectroscopy, IR spectroscopy, mass spectroscopy andNMR spectroscopy analyses were performed.

(1) General Properties

HNP-98701A, HNP-98701B and HNP-98701C are all white and odorless.

(2) Solubility

HNP-98701A, HNP-98701B and HNP-98701C are highly soluble in methanol, ethanol, ethyl acetate, chloroform and, dimethylsulfoxide (DMSO), but insoluble in water, acetone, ether and hexane.

(3) UV Spectroscopy

HNP-98701A, HNP-98701B and HNP-98701C all have UV absorption bands at 230 nm and 280 nm.

(4) IR Spectroscopy

HNP-98701A, HNP-98701B and HNP-98701C have identical IR absorption spectrum. To be specific, these compounds have absorption bands at wavenumbers 3,442 cm⁻¹, 2,962 cm⁻¹, 2,927 cm⁻¹, 1,652 cm⁻¹, 1,607 cm⁻¹, 1,511 cm⁻¹, 1,418 cm⁻¹, 1,264 cm⁻¹, 1,138 cm⁻¹, 1,029 cm⁻¹, 855 cm⁻¹, 810 cm⁻¹ and 750 cm⁻¹. The absorption band at 3,442 cm⁻¹ is due to hydrogen-bonded hydroxyl group (OH), and the absorption bands at 2,962 cm⁻¹ and 2,927 cm⁻¹ are due to —CH. The absorption bands at 1,652 cm⁻¹, 1,607 cm⁻¹ and 1,418 cm⁻¹ are due to double bond (C═C) of aromatic rings; the absorption band at 1,511 cm⁻¹ is due to the substituted functional group other than hydrogen (H) at 1-, 2- and 4-positions of aromatic ring; and the absorption bands at 1,138 cm⁻¹ and 1,029 cm⁻¹ are due to C—O. And, the absorption bands at 855 cm⁻¹, 810 cm⁻¹ and 750 cm⁻¹ are due to out-of-plane bending of ═CH of aromatic ring.

(5) Mass Spectroscopy

Although molecular weight of HNP-98701A, HNP-98701B and HNP-98701C in FAB+ mass spectroscopy is 754, which is different from that of NMR spectroscopy, that of EI+ mass spectroscopy is 732, which is identical to that of NMR spectroscopy.

(6) NMR Spectroscopy

NMR spectroscopy results for H-NMR and C-NMR about HNP-98701A and HNP-98701C are shown in the following Table 1. Results of HNP-98701B, HNP-98701A and HNP-98701C are identical.

TABLE 1
Position of Hydrogens and Carbons

| | δH | | δC | |
|---|---|---|---|---|
| | HNP- | HNP- | HNP- | HNP- |
| | 98701A | 98701C | 98701A | 98701C |
| C-2 | 5.48(d, 5.88 Hz) | 5.47(d, 5.84 Hz) | 83.4 | 83.4 |
| C-3 | 2.31(m) | 2.31(m) | 44.3 | 44.2 |
| C-4 | 2.31(m) | 2.31(m) | 44.3 | 44.2 |
| C-5 | 5.48(d, 5.88 Hz) | 5.47(d, 5.84 Hz) | 83.4 | 83.4 |
| C-6 | 0.72(d, 6.54 Hz) | 0.73(d, 6.3 Hz) | 14.9 | 14.9 |
| C-7 | 0.72(d, 6.54 Hz) | 0.73(d, 6.3 Hz) | 14.9 | 14.9 |
| C-1′ | — | — | 145.5 | 146.5 |
| C-2′ | 6.83(d, 8.18 Hz) | 6.83(d, 8.11 Hz) | 118.5 | 118.7 |
| C-3′ | 6.86(d) | 6.94(d, 8.11 Hz) | 110.8 | 110.0 |
| C-4′ | — | — | 132.5 | 132.6 |
| C-5′ | — | — | 148.9 | 149.0 |
| C-6′ | 6.86(d, 8.18 Hz) | 6.93(d, 8.12 Hz) | 119.0 | 120.0 |
| C-1″ | — | — | 136.7 | 136.5 |
| C-2″ | 6.93(d. 8.17 Hz) | 6.84(d, 8.12 Hz) | 110.2 | 110.9 |
| C-3″ | 7.02(d, 8.17 Hz) | 6.99(d, 8.12 Hz) | 119.6 | 118.7 |
| C-4″ | — | — | 148.2 | 148.9 |
| C-5″ | — | — | 151.3 | 150.6 |
| C-6″ | 6.99(s) | 6.92(s) | 109.6 | 110.1 |
| C-1″′ | 4.37(m) | 4.15(m) | 82.5 | 84.1 |
| C-2″′ | 4.85(d, 2.96 Hz) | 4.65(d, 8.28 Hz) | 73.6 | 78.4 |
| C-3″′ | 1.18(d, 6.38 Hz) | 1.17(d, 6.24 Hz) | 13.5 | 17.1 |
| OCH3 | | | | |

The present invention provides extracts and separated substances of novel compounds HNP-98701A, HNP-98701B and known compound HNP-98701C obtained from saururus, which have anticancer activity.

In order to attain this object, saururus extract obtained by extracting saururus with low alcohol, saururus organic-solvent extracted fraction obtained by extracting the saururus extract with organic solvents like hexane, chloroform, ethyl acetate and butanol, and substance obtained by separating the saururus organic-solvent extracted fraction with silica gel-filled column chromatography were identified to have anticancer activity.

The present invention also provides substances obtained by decomposing the novel compounds HNP-98701A and HNP-98701B and known compound HNP-98701C with acid or base. These decomposed substances also have anticancer activity.

The present invention also provides a preparation method of novel compounds HNP-98701A and HNP-98701B and known compound HNP-98701C by extracting saururus.

This preparation method comprises:

i) A step of immersing saururus in low alcohol and repeatedly extracting it;

ii) A step of extracting the low alcohol extract by adding various organic solvents;

iii) A step of separating the various organic solvent extracted fractions with chromatography to obtain carcinostatis substance; and

iv) A step of purifying each carcinostatis substance.

Lastly, the present invention provides pharmaceutical composition containing novel compounds HNP-98701A and HNP-98701B and known compound HNP-98701C, or mixture thereof (HNP-98701) as effective constituent.

Hereunder is given a more detailed description of the present invention.

First, saururus extract with anticancer activity is separated from saururus.

For this purpose, we immersed saururus in low alcohol and repeatedly extract it to obtain alcohol fraction of saururus having anticancer activity. For saururus, it is recommended to use residues remaining after using saururus as live herb, dried herb, stem top, root and tea, including wildly grown or cultured saururus; and for alcohol, it is recommended to use methanol.

The saururus extract is re-extracted with organic solvent to separate saururus extract having anticancer activity.

To be specific, we extracted saururus with methanol and concentrated it. Then, using various organic solvents, we prepared various solvent-extracted saururus fractions with superior anticancer activity by fractionating it.

Extraction using organic solvent is performed in the order from nonpolar solvent to polar solvent, and for organic solvent, low alcohol, ethyl acetate, hexane, chloroform or butanol can be used. As a preferred embodiment of the present invention, using n-hexane, chloroform, ethyl acetate and butanol in order, solvent-extracted fraction and aqueous fraction were prepared for each solvent. Among them, saururus extract obtained by ethyl acetate showed the highest anticancer activity. The extraction temperature is recommended to be 4-100° C., and more preferably to be room temperature or 4-30° C.

In order to separate carcinostatis substance from the organic solvent-extracted fraction using column chromatography filled with silica gel, we concentrated the ethyl acetate extracted fraction and dissolved it in methanol or ethyl acetate. Then, after dropping it in column chromatography filled with silica gel, it was eluted with organic solvent mixture comprising n-hexane, ethyl acetate and methanol with various compositions. Then, it was separated by thin-layer chromatography to obtain carcinostatis substance.

The thin-layer chromatography analysis result showed the carcinostatis substance located between 0.01-0.5 of run of flow (R_{f}). This substance was collected and repeatedly concentrated to separate the carcinostatis substance. This compound was named as HNP-98701.

Then, HNP-98701A, HNP-98701B and HNP-98701C are separated from HNP-98701 using prep-thin-layer chromatography (prepTLC).

For this purpose, we concentrated the HNP-98701 faction and dissolved it in methanol or ethyl acetate. Then, after dropping it in prep-thin-layer chromatography coated with silica gel, it was eluted with organic solvent mixture comprising n-hexane, ethyl acetate and methanol. The prep-thin-layer chromatography analysis result showed substances located at three regions between R_{f} 0.2-0.9. We collected each substance and repeatedly concentrated it to obtain three kinds of carcinostatis substances. They were named as HNP-98701A, HNP-98701B and HNP-98701C.

In order to further purify the obtained HNP-98701A, HNP-98701B and HNP-98701C, we removed yellow pigment by solvent precipitation using solubility difference in n-hexane and ethyl acetate to prepare white carcinostatis substances HNP-98701A, HNP-98701B and HNP-98701C. Purity of each substance was identified to be very superior by high pressure liquid chromatography (HPLC).

Lastly, the present invention provides carcinostatis pharmaceutical composition containing carcinostatis substances expressed by Formulas 1-3, or mixture thereof and derivatives thereof as effective constituent.

The compounds expressed by Formulas 1-3 and derivatives thereof may be administered orally or non-orally in the form of muscular or intravenous injection, and it can be used together with the conventional antitumor medicine in the form of general medicinal drug.

That is, the compounds expressed by Formulas 1-3 and their derivatives can be administered orally or non-orally. They can be made in drug form with the commonly used diluent or vehicle, such as filler, extender, binder, wetting agent, disintegrator and surfactant. Tablet, pill, powder, granule and capsule are included in solid drug form for oral administration. These solid drugs are prepared by mixing the extracts expressed by Formulas 1-3 with more than one vehicles, such as starch, calcium carbonate, sucrose or lactose and gelatin. Also, lubricants like magnesium stylate talc can be used in addition to vehicles. Suspension, internal medicine, emulsion and syrup are included in liquid drug form for oral administration. Diluents like water and liquid paraffin and various vehicles like wetting agent, sweetener, aromatic and preservative can be included. Sterile aqueous solution, nonaqueous solvent, suspension, emulsion, lyophilizator and suppository are included in drug form for non-oral administration. For nonaqueous solvent or suspension, propylene glycol, polyethylene glycol, plant oil like olive oil and injectable ester like ethyl olate can be used. For suppository, witepsol, macrogol, tween 61, cacao oil, lauryn oil or glycerogelatin can be used.

For effective constituent of the pharmaceutical composition, compounds expressed by Formulas 1-3, mixture thereof or derivatives thereof can be used in 0.1-3.0 mg/kg, and preferably in 0.3-11.0 mg/kg. It can be administered once a day.

While the compounds expressed by Formulas 1-3 do not have toxicity against cell line of normal cell, they selectively affect cancer cell line of liver cancer and prostate cancer and induce cell death. Therefore, carcinostatis pharmaceutical composition containing it as effective constituent can be used for treatment of liver cancer, breast cancer, throat cancer, melanosis, lung cancer, prostate cancer, rectal cancer, stomach cancer, cervical cancer, esophageal cancer, tongue cancer, oral cancer, pancreas cancer, thyroid cancer, leukemia and myeloma, and preferably for treatment of liver cancer, prostate cancer, breast cancer, throat cancer, melanosis and stomach cancer, and more preferably for treatment of liver cancer and prostate cancer.

Besides, the pharmaceutical composition containing the compounds expressed by Formulas 1-3 and derivatives thereof effective constituent as effective constituent can be used for functional food or food additive, functional drink or drink additive, carcinostatis cosmetic additive, carcinostatis soap additive and carcinostatis shampoo additive.

### BRIEF DESCRIPTION OF THE DRAWINGS

A more complete appreciation of the present invention, and many of the attendant advantages thereof, will become readily apparent as the same becomes better understood by reference to the following detailed description when considered in conjunction with the accompanying drawings in which like reference symbols indicate the same or similar components, wherein:

FIG. 1 represents the anticancer activity of HNP-98701 in terms of tumor size, for nude mouse wherein prostate cancer cell line (DU-145) was transplanted hypodermically.

### DETAILED DESCRIPTION FOR PREFERRED EMBODIMENT

It should be understood that the description of the preferred embodiment is merely illustrative and that it should not be taken in a limiting sense. In the following detailed description, several specific details are set forth in order to provide a thorough understanding of the present invention. It will be obvious, however, to one skilled in the art that the present invention may be practiced without these specific details.

### EXAMPLE 1

### Preparation of Methanol Extract from Saururus

100 g of dried saururus powder was immersed in 1L of methanol for 3 days at room temperature. After filtering this extract, the residue was extracted again with methanol. This procedure was repeated for 3 times. The obtained filtrate was concentrated at 20-60° C. to obtain 8 g of methanol extract.

The obtained methanol extract was dissolved in dimethylsulfoxide (hereunder referred to as “DMSO”), and its anticancer activity was identified from MTT analysis of Experimental Example 1 (Table 2).

### EXAMPLE 2

### Preparation of Various Organic Solvent Extracted Fractions From Methanol Extract

<2-1> Preparation of Hexane Extracted Fraction

5 g of methanol extract prepared in Example 1 was suspended in 100 ml of distilled water. After inserting this solution and 100 ml of n-hexane in 500 ml of fraction funnel, the mixture was stirred and let alone for 5 hr. Then, the n-hexane layer was collected. This procedure was repeated for 4 times. The obtained n-hexane layer was collected, filtered and concentrated to obtain 0.8 g of hexane extracted fraction.

The obtained hexane extracted fraction was dissolved in DMSO, and its anticancer activity was identified from MTT analysis of Experimental Example 1 (Table 2).

TABLE 2
Anticancer Activity Determination of Each Solvent Extract against
Liver Cancer Cell Line: IC₅₀ (μg/ml)

| Sample | SK-Hep-1 |
|---|---|
| Methanol extract | 1.0970 |
| Hexane extract | 2.5564 |
| Ethyl acetate extract | 0.3090 |
| Butanol extract | 2.0612 |
| Water Extract | >50 |
| Cisplatin | 5.4350 |
| 5-FU | >50 |

<2-2> Preparation of Ethyl Acetate Extracted Fraction

After adding 100 ml of ethyl acetate to 100 ml of water layer remaining after preparing the hexane extracted fraction in Example 2, the solution was stirred and let alone for 5 hr. Then, the ethyl acetate layer was collected. This procedure was repeated for 4 times. The obtained ethyl acetate layer was collected, filtered and concentrated to obtain 1.7 g of ethyl acetate extracted fraction.

The obtained ethyl acetate extracted fraction was dissolved in DMSO, and its anticancer activity was identified from MTT analysis of Experimental Example 1 (Table 2).

### <2-3> Preparation of Butanol Extracted Fraction

After adding 100 ml of butanol to 100 ml of water layer remaining after preparing the ethyl acetate extracted fraction in Example 3, the solution was stirred and let alone for 5 hr. Then, the butanol layer was collected. This procedure was repeated for 4 times. The obtained ethyl acetate layer was collected, filtered and concentrated to obtain 3.6 g of butanol extracted fraction.

The obtained butanol extracted fraction was dissolved in DMSO, and its anticancer activity was identified from MTT analysis of Experimental Example 1 (Table 2).

<2-4> Preparation of Water Extracted Fraction

100 ml of water layer remaining after preparing the butanol extracted fraction in Example 4 was filtered and concentrated to obtain 1.9 g of water extracted fraction.

The obtained water extracted fraction was dissolved in DMSO, and its anticancer activity was identified from MTT analysis of Experimental Example 1 (Table 2).

### EXAMPLE 3

### Separation of Carcinostatis Substance HNP-98701 From Ethyl Acetate Extracted Fraction by Column Chromatography

Among the organic solvent extracted fractions prepared in Example 2, 1 g of ethyl acetate extracted fraction, which shows the best anticancer activity, was dissolved in 2 ml of ethyl acetate. Then, it was inserted to the top of column where in silica gel (Merck Co.) is filled. After covering with seashore sand, it was eluted with organic solvents. For the elution solvents, n-hexane, 2:1, 1:1, 2:3, 1:2 and 1:4 composition of n-hexane:ethyl acetate were used in order, and it was eluted again with ethyl acetate, 20:1 and 10:1 composition of ethyl acetate: methanol in order. Anticancer activity of each eluted fraction was identified from MTT analysis of Experimental Example 1, and the active fraction having anticancer activity was repeatedly eluted through second, third and fourth column chromatography. Using elution solvent comprising n-hexane:ethyl acetate=1:2-1:4, yellow substance having anticancer activity was separated. We named this compound as HNP-98701.

Each separated fraction was dissolved in DMSO, and its anticancer activity was identified from MTT analysis of Experimental Example 1 (Table 3).

TABLE 3
Anticancer Activity Determination of Each Eluted
Active Substance Separated by Column
Chromatography against Liver Cancer Cell
Line: IC₅₀ (μg/ml)

| Sample | SK-Hep-1 |
|---|---|
| Column 1 | 0.0927 |
| Column 2 | 0.0556 |
| Column 3 | 0.0487 |
| Column 4 | 0.0455 |
| Cisplatin | 4.2950 |
| Adriamycin | 0.2817 |

### EXAMPLE 4

### Separation of Carcinostatis Substances HNP-98701A, HNP-98701B and HNP-98701C from HNP-98701 by Thin-Layer Chromatography

70 mg of HNP-98701 separated by column chromatography in Example 3 was dissolved in 0.5 ml of ethyl acetate. After dropping this solution on prep-TLC (Merck Co.), it was repeatedly developed using 1:2 composition of n-hexane:ethyl acetate as developing solvent. R_{f} of HNP98701 was divided at three points between 0.2 and 0.9. The compounds were named as HNP-98701A, HNP-98701B and HNP-98701C in order of R_{f} value. Each band was collected and dried respectively to obtain pale yellow compounds HNP-98701A, HNP-98701B and HNP-98701C.

### EXAMPLE 5

### Purification of Carcinostatis Substances HNP-98701A, HNP-98701B and HNP-98701C Using Solvent Precipitation

10 mg of each pale yellow compound HNP-98701A, HNP-98701B and HNP-98701C separated in Example 4 was dissolved in 1 ml of ethyl acetate. Precipitate was generated by adding n-hexane little by little. After letting this mixture solution in refrigerator overnight, it was filtered to obtain white precipitate of compounds HNP-98701A, HNP-98701B and HNP-98701C.

### EXAMPLE 6

### Purity Determination of Carcinostatis Substances HNP-98701A, HNP-98701B and HNP-98701C

Purity of white carcinostatis substances HNP-98701 A, HNP-98701B and HNP-98701C solvent precipitated in Example 5 was determined by high performance liquid chromatography (hereunder referred to as “HPLC”).

To be specific, 10 mg of each compound was dissolved in 1 ml of methanol. These samples were inserted in hypersil-BDS C18 column (Hewlett Packard), and separated with methanol and distilled water as mobile phases. Gradually changing concentration of the mixture solution from 100% distilled water to 100% methanol at the rate of 0.5 ml/min for 50 min, by 2% per minute, the absorption band was observed. At 30 min, a highly outstanding single absorption band was identified, which shows that these compounds are very pure.

The separated carcinostatis substances HNP-98701A, HNP-98701B and HNP-98701C were dissolved in DMSO, and their anticancer activities were identified from MTT analysis of Experimental Example 1. Also, the mixture HNP-98701 comprising these three compounds was dissolved in DMSO, and used in animal toxicity of Experimental Example 2 (Tables 4, 5, 6 and 7).

TABLE 4
Anticancer Activity Determination of Compounds
HNP-98701A, HNP-98701B and HNP-98701C
Separated by High Performance Liquid
Chromatography against Liver Cancer
Cell Line: IC₅₀ (μg/ml)

| Sample | SK-Hep-1 |
|---|---|
| HNP-98701A | 0.0795 |
| HNP-98701B | 0.0388 |
| HNP-98701C | 0.0560 |
| Cisplatin | 4.2950 |
| Adriamycin | 0.2817 |

TABLE 5
Anticancer Activity Determination of Compounds
HNP-98701A, HNP-98701B and HNP-98701C
Separated by High Performance Liquid
Chromatography against Prostate
Cancer Cell Line: IC₅₀ (μg/ml)

| Sample | PC-3 | DU-145 |
|---|---|---|
| HNP-98701A | 0.1249 | 0.0537 |
| HNP-98701B | 0.0695 | 0.0184 |
| HNP-98701C | 0.0446 | 0.0263 |
| Cisplatin | 2.4867 | 3.0012 |
| Adriamycin | 0.5752 | >50 |

TABLE 6
Anticancer Activity Determination of Compounds HNP-98701A,
HNP-98701B and HNP-98701C Separated by High Performance Liquid
Chromatography against Various Human c Cancer Cell Line: IC₅₀ (μg/ml)

| | Derivation from | | | | | |
|---|---|---|---|---|---|---|
| | Human Cancer | | HNP- | HNP- | HNP- | |
| Cell Line | Cell Line | Cisplatin | Adriamycin | 98701A | 98701B | 98701C |
| SK-Hep-1 | liver, | 4.2950 | 0.2817 | 0.0795 | 0.0388 | 0.0560 |
| | adenocarcinoma | | | | | |
| PC-3 | prostate, | 2.4867 | 0.5752 | 0.1249 | 0.0695 | 0.0446 |
| | adenocarcinoma | | | | | |
| DU-145 | prostate, | 3.0012 | >50 | 0.0537 | 0.0184 | 0.0263 |
| | carcinoma | | | | | |
| BT-20 | breast, carcinoma | 1.4578 | 0.1312 | 0.0996 | 0.0292 | 0.0629 |
| BT-549 | breast, | 1.2522 | 0.4382 | *^{a} | * | * |
| | ductal carcinoma | | | | | |
| HS-578T | breast, | 2.3317 | 0.4685 | N.E.^{b} | N.E. | N.E. |
| | ductal carcinoma | | | | | |
| MCF-7 | breast, carcinoma | 2.9412 | 0.1342 | >10 | >10 | >10 |
| SK-BR-3 breast, | 1.1748 | 0.7052 | 0.1698 | 0.0866 | 0.0632 | |
| | adenocarcinoma | | | | | |
| T-47D | breast, | 2.0468 | 0.2548 | 0.0400 | 0.0167 | 0.1249 |
| | ductal carcinoma | | | | | |
| HeLa | cervix, epithelial | 5.3358 | 0.7028 | 0.4233 | 0.1680 | 0.1838 |
| | carcinoma | | | | | |
| ME-180 | cervix, carcinoma | 0.9365 | 0.1077 | * | * | * |
| HT-29 | colon, | 6.1224 | 0.4127 | * | * | * |
| | adenocarcinoma | | | | | |
| LoVo | colon, | 0.7318 | 0.0538 | * | * | * |
| | adenocarcinoma | | | | | |
| SK-N-SH | neuroblastoma | 2.1178 | 0.2070 | 1.2849 | 0.4314 | 0.5392 |
| T98G | glioblastoma | 7.9216 | 1.6132 | 0.2470 | 0.1003 | 0.1014 |
| HS-746T | stomach, | 2.7680 | >50 | * | * | * |
| | carcinoma | | | | | |
| KATO-III | gastric carcinoma | 2.2875 | 0.3415 | 0.6171 | 0.4477 | 0.7005 |
| A431 | epidermoid | 0.4240 | 0.0675 | 0.4903 | 0.2344 | 0.3860 |
| | carcinoma | | | | | |
| Hep-2 | larynx | 0.5518 | 0.0870 | 0.0406 | 0.0329 | 0.0367 |
| | epidermoid | | | | | |
| | carcinoma | | | | | |
| HT1080 | fibrocarcinoma | 2.9512 | 0.7192 | 0.4267 | 0.1882 | 0.1753 |
| NCI-H69 | small cell lung | 0.6382 | 0.1675 | * | * | * |
| | carcinoma | | | | | |
| SK-MEL- | melanoma, | 4.3157 | 0.7498 | 0.2544 | 0.1123 | 0.0725 |
| 28 | malignant | | | | | |
| SK-OV-3 | ovary | 5.3442 | >50 | N.E. | N.E. | N.E. |
| | adenocarcinoma | | | | | |
| HS683 | glioma | 0.2490 | 0.2051 | 0.5561 | 0.2325 | 0.2170 |

*: Existence rate larger than 60% for 10 μg/ml sample
N.E.: Not effective; existence rate larger than 90% for 10 μg/ml sample

TABLE 7
Cytotoxicity Determination of Compounds HNP-98701A, HNP-
98701B and HNP-98701C Separated by High Performance Liquid
Chromatography against Various Normal Cell Lines: IC₅₀ (μg/ml)

| | Derivation of Cell | | | HNP- | HNP- | HNP- |
|---|---|---|---|---|---|---|
| Cell Line | Line | Cisplatin | Adriamycin | 98701A | 98701B | 98701C |
| 293 | transformed | 1.1678 | <0.008 | *^{a} | * | * |
| | embryonal kidney, | | | | | |
| | human | | | | | |
| 3T3-L1 | embryo, mouse | 2.7096 | 1.9982 | N.E.^{b} | N.E. | N.E. |
| CHO | ovary, Chinese | 2.8462 | 5.2223 | * | * | * |
| | hamster | | | | | |
| CPAE | pulmonary artery | 0.8252 | 0.0208 | * | * | * |
| | endothelium, | | | | | |
| | bovine | | | | | |
| HS27 | newborn foreskin, | 1.7853 | 0.2178 | N.E. | N.E. | N.E. |
| | human | | | | | |
| NIH-3T3 | contact-inhibited | 1.9756 | 0.4963 | N.E. | N.E. | N.E. |
| | embryo, mouse | | | | | |

*: Existence rate larger than 60% for 10 μg/ml sample
N.E.: Not effective; existence rate larger than 90% for 10 μg/ml sample

### EXAMPLE 7

### Preparation of Carcinostatis Substance HNP-98701C Derivatives

<7-1> Preparation of Acetyl Derivative

After adding acetic anhydride (Ac₂O) and pyridine to 15.9 mg of the carcinostatis substance HNP-98701C purified in Example 5, it was reacted for 8 hr at room temperature. After dissolving the product in ethyl acetate, it was developed with 2:1 composition of ethyl acetate/hexane developing solvent. Unreacted substance (OAc-s), acetyl derivative with only one —OH group reacted (OAc-1) and acetyl derivative with both —OH groups reacted (OAc-2) were obtained in 4.2 mg, 6.9 mg and 3.0 mg, respectively, by prep-TLC. Anticancer activity of each acetyl derivative was identified from MTT analysis of Experimental Example 1 (Table 8).

TABLE 8
Anticancer Activity Determination of Acetyl Derivative and Methyl
Derivative of Compound HNP-98701C against Cancer Cell Lines: IC₅₀ μg/ml)

| Cell | | | | | | | |
|---|---|---|---|---|---|---|---|
| Line | Cisplatin | Adriamycin | HNP-98701C | OAc-1 | OAc-2 | OMe-1 | OMe-2 |
| SK- | 4.2950 | 0.2817 | 0.0560 | 0.5736 | 0.6448 | 0.6165 | 4.9782 |
| Hep-1 | | | | | | | |
| PC-3 | 2.4867 | 0.5752 | 0.0446 | 0.5346 | 0.6698 | 0.4988 | 3.5765 |
| DU-145 | 3.0012 | >50 | 0.0263 | 0.4654 | 0.6160 | 0.3785 | 2.8876 |

<7-2> Preparation of Methyl Derivative

After adding NaH and CH₃I to 17.7 mg of the carcinostatis substance HNP-98701C purified in Example 5, it was reacted for 8 hr at room temperature. After dissolving the product in ethyl acetate, it was developed with 2:1 composition of ethyl acetate/hexane developing solvent. Unreacted substance (OMe-s), methyl derivative with only one —OH group reacted (OMe-1) and methyl derivative with both —OH groups reacted (OMe-2) were obtained in 8.5 mg, 7.0 mg and 1.9 mg, respectively. Anticancer activity of each methyl derivative was identified from MTT analysis of Experimental Example 1 (Table 8).

### EXPERIMENTAL EXAMPLE 1

### Anticancer Activity Determination Using MTT Analysis

MTT analysis was performed in order to determine anticancer activity of each extracted and separated substance in Examples 1-7.

To be specific, SK-Hep-1 cell, which is liver cancer cell line, was added to 96-well microplate (Falcon, USA) with the concentration of 3×10⁵ cells/ml by 100 μl per well. Each extracted and separated substance dissolved in DMSO was added to the well by 100 μl for the final concentration to be from 50 μg/ml to 0.0244 μg/ml, with 200 μl of final volume. Each cell was cultured for 48 hr in a culture medium of 37° C. and 5% CO₂. After cultivation, 20 μl of phosphate buffer solution containing 5 mg/ml of MTT (3-[4,5-dimethylthiazol-2-yl]-2,5-diphenyltetrazoliumbromide, Sigma Co.) reagent was added to each well, and then cultured for 4 hr. After taking 100 μl of supernant from each culture medium, 100 μl of 0.04N hydrochloric acid-isopropanol was added. After letting alone overnight, absorbance was measured at 560 nm using automatic Eliza detector (Molecular Divices Corp., USA). Anticancer activity of each extracted and separated substance was calculated from the absorbance value by Equation 1 and was expressed in terms of cell.

<Equation 1>

Average Existence Rate(%)=(Absorbance of Treated Group)/(Absorbance of Control Group)×100

Among each extracted and separated substance, anticancer activity against liver cancer cell line was examined for the samples that have average existence rate larger than 50%. The result was that the extracted and separated substances of the present invention have superior anticancer activity compared to the conventional liver cancer treatments like cisplatin, 5-fluorouracil (5-FU) and adriamycin (doxorubicin) (Refer to Table 2, Table 3, Table 4, Table 5, Table 6, Table 7 and Table 8).

### EXPERIMENTAL EXAMPLE 2

### Anticancer Activity Determination of Compound HNP-98701 Through Animal Experiment

As shown in Table 4 and Table 5, the carcinostatis substances HNP-98701A, HNP-98701B and HNP-98701C of the present invention showed very superior cell death effect against liver cancer cell line and prostate cancer cell line. In order to identify anticancer activity of the mixture (HNP-98701) of the compounds HNP-98701A, HNP-98701B and HNP-98701C, animal experiment was performed.

To be specific, liver cancer cell line (SK-Hep-1) and prostate cancer cell line (DU-145) were transplanted hypodermically to the back of nude mouse (balb/c athymic nude mouse). When the tumor size reached 150-200 mm³, HNP-98701 was injected intraperitoneally (ip). The volume of tumor was measured between 2 weeks and 5 weeks after injection, according to Equation 2. The tumor tissue was bisected, dyed and then observed with a microscope.

<Equation 2>

Volume of Tumor=½ ab²

In Equation 2, a is the longer diameter of the tumor, and b is the shorter diameter.

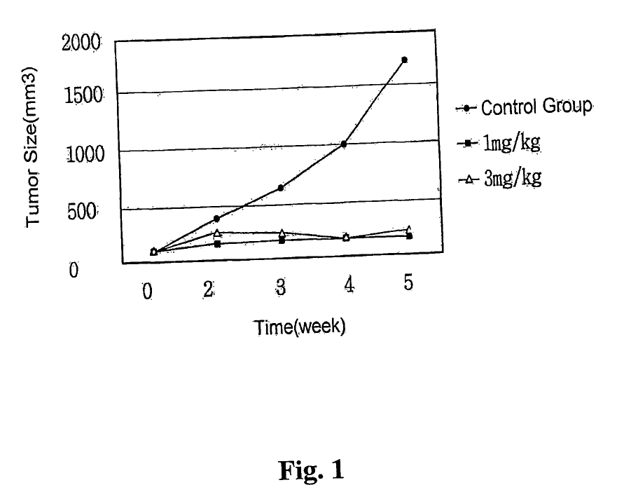

As a result, the compound HNP-98701 of the present invention showed 89% of tumor propagation inhibition effect in case of 1 mg/kg administration, and 85% of tumor propagation inhibition effect in case of 3 mg/kg administration for prostate cancer (FIG. 1). Also, microscopic observance showed that the compound HNP-98701 of the present invention induced necrosis of inner tissue of the tumor.

On the other hand, anticancer activity could not be compared for liver cancer because the tumor of HNP-98701-untreated group did not grow larger than 200-250 mm³. However, microscopic observance showed that HNP-98701-treated group had leukocyte membrane outside of the tumor. This implies that the compound HNP-98701 affect cancer cell and facilitates attack of leukocyte to induce cell death. Therefore, the compound HNP-98701 of the present invention is very effective in prostate cancer and liver cancer.

### EXPERIMENTAL EXAMPLE 3

### Examination of Compound HNP-98701-Induced Cell Death Type by DNA Fragmentation Extraction

Cell death is divided roughly into apoptosis and necrosis. Apoptosis is a inner cell death caused by signal transfer due to stimulus from outside of the cell, and necrosis is destruction of cell caused by abrupt environmental change outside the cell or impact on the cell. Therefore, if the compound HNP-98701 of the present invention causes apoptosis, its availability as carcinostatis substance will be enhanced greatly.

In general, cell death type is identified by extracting DNA from treated cell and performing electrophoresis to identify the DNA pattern. If a regular ladder-shaped DNA pattern is obtained, the cell death is due to apoptosis, and otherwise if the DNA pattern is random, it is due to necrosis.

Based on this fact, DNA fragmentation extraction was performed to identify cell death type caused by the carcinostatis substance HNP-98701 of t h e present invention. To be specific, 3×10⁶ cells/ml of U937 cell was prepared in 10 ml. It was treated with 10 μg/ml, 5 μg/ml and 1 μg/ml of HNP-98701. For control groups, non-treated group, the one treated with 10 μg/ml of Cisplatin and the one treated with 10 μg/ml of adriamycin were used. All the groups were cultured at 37° C. for 24 hr. Then, each cell was centrifuged and collected in a test tube. After washing with phosphate buffer solution, 500 μl of lysis buffer solution(0.25% NP-40 in TBE buffer) was added to each cell. After treating RNase (100 μg/ml) and proteinase (proteinase K, 0.1-1μ/ml) in the cell solution and reacting it for 30 min at 37° C., DNA was separated by phenol/chloroform extraction and ethanol precipitation. The DNA was electrophoresed in 1.3-1.5% agarose gel to identify the DNA pattern.

As a result, DNA of the cell treated by the compound HNP-98701 of the present invention showed ladder pattern, which is typical of apoptosis. This implies that HNP-98701 induces apoptosis-type cell death.

### EXPERIMENTAL EXAMPLE 4

### Effect of Compound HNP-98701 on Cell Cycle

In order to identify intra-cell signal transfer mechanism of the compound HNP-98701 of the present invention, kinase reaction activity of HNP-98701 against cell-cycle-related component was examined. For the cell-cycle-related components, there are CDK2, which participates in G1-S phase, CDK4, which participates in G1 phase, and Cdc2 (CDK1), which participates in G2-M phase. If kinase activity of these components is inhibited, normal cell division does not proceed and cell apoptosis is induced. Accordingly, by examining kinase activity of CDKs of cell treated with the compound HNP-98701 of the present invention, one can know which phase of cell cycle HNP-98701 takes part in.

In general, kinase activity is identified with two methods. The first method is to separate CDKs from cell and identify the kinase activity of CDKs themselves. The second method is to treat the cell with sample and separate CDKs to identify amount of CDKs remaining in cell.

<4-1> Determination of CDK2 Kinase Activity (Chemical Treatment of Separated CDK2)

Firstly, we identified CDK2 kinase activity of G1-S phase in order to know if HNP-98701 participates in G1-S phase and causes cell death.

To be specific, L929 cell, which is hypodermic connective tissue cell line of mouse, and 293 cell, which is embryo-modified kidney cell line, were sufficiently cultured on 100-mm tissue culture dish. Then, it was washed with phosphate buffer solution for two times. After lyzing the cells by adding 1 me of RIPA buffer solution (50 mM tris-HCl, pH 7.5, 150 mM NaCl, 1% NP-40, 0.5% deoxycholic acid, 0.1% SDS, 1 mM PMSF), cell debris was removed by centrifuge. From the solution containing cellular protein, protein was quantized. After adding anti-Cdk2 antibody (rabit polyclonal IgG, Upstate Biotechnology Inc.) in 1-10 μg per 3 mg of protein to the solution, it was mixed carefully at 4° C. for 1 hr. After adding 15 μl of protein-G agarose (Santa Cruz Biotechnology Inc.) to the solution, it was mixed at the same condition and then centrifuged. After collecting Cdk2-bound protein-G agarose in test tube, it was washed with RIPA buffer solution twice and with 20 mm HEPES buffer solution thrice to prepare immunoprecipitate to be used for kinase activity reaction. After adding a suitable amount of HEPES buffer solution to the immunoprecipitate, it was transferred to test tube, and reaction buffer solution and histone H-1 (4 μg, Sigma Co.) substrate were added. Then, HNP-98701 was added for the final concentration to be 100 μM. After adding 1 mCi of γ³²-ATP (Amersham) to the mixture solution, kinase reaction was performed for 30 min at 30° C. The reaction was terminated by adding stop buffer and heating the solution. This reaction mixture was electroporesed in 12% SDS PAGE and developed. After drying gel, it was exposed to X-ray film to identify the band pattern.

As a result, while the compound HNP-98701 of the present invention caused no effect on CDK2 kinase activity of L929 cell, it reduced CDK2 kinase activity of 293 cell (Table 9).

TALBE 9
Determination of Reaction Inhibition Activity
of Compound HNP-98701 against CDK2
Kinase of L929 and 293 Cell Line

| | Sample Treatment | | |
|---|---|---|---|
| Cell Line | Concentration(μM) | Band Absorbance | Absorbance (%) |
| L929 | — | 0.891 | 100 |
| L929 | 100 | 0.834 | 93.6 |
| 293 | — | 0.630 | 100 |
| 293 | 100 | 0.435 | 69.0 |

<4-2> Reaction Inhibition Activity of Compound HNP-98701 Against Various CDK2 Cell Lines (Determination of CDK2 Activity After Chemical Treatment of Cell Lines)

Kinase reaction activity was determined as in <4-1>.

To be specific, various cell lines including L929 293, SK-Hep-1 liver cancer cell line and PC3 prostate cancer cell line were cultured on 100 mm tissue culture dish to about 70-80%. After adding 5 ml of 0.2 μg/ml, 0.3 μg/ml and 1 μg/ml HNP98701, the cell lines were cultured for 24 hr, and then washed with phosphate buffer solution for two times. Then, after lyzing the cells to prepare immunoprecipitate, kinase reaction activity was induced as in <4-1>.

As a result, while the compound HNP-98701 of the present invention caused no effect on the amount of CDK2, it reduced the amount of CDK2 of 293, SK-Hep-1 and PC3 cells, as in <4-1>. Therefore, the compound HNP-98701 of the present invention was identified to induce apoptosis-type cell death in 293, SK-Hep-1 and PC3 cells. That is, it inhibits CDK2 kinase acitivity that participates in G1-S phase of cell cycle (Table 10).

TABLE 10
Determination of Reaction Inhibition Activity of
Compound HNP-98701 against CDK2 Kinase
of Various Cell Lines

| | Sample Treatment | Sample | | |
|---|---|---|---|---|
| | Concentration | Treatment | Band | Absorbance |
| Cell Line | (μg/ml) | Time (hr) | Absorbance | (%) |
| L929 | — | — | 1.313 | 100.0 |
| L929 | 1 | 24 | 1.330 | 101.3 |
| 293 | — | — | 0.882 | 100.0 |
| 293 | 1 | 24 | 0.335 | 37.9 |
| SK-Hep-1 | — | — | 0.491 | 100.0 |
| SK-Hep-1 | 0.2 | 24 | 0.148 | 30.1 |
| SK-Hep-1 | 1 | 24 | 0.253 | 51.5 |
| PC-3 | — | — | 0.296 | 100 |
| PC-3 | 0.3 | 24 | 0.045 | 15.2 |
| PC-3 | 1 | 24 | 0.088 | 29.7 |

<4-3> Determination of CDK4 Kinase Reaction Activity

We determined CDK4 kinase activity of G1 phase in order to know if HNP-98701 participates in G1 phase and causes cell death.

The same procedure as that of <4-1> for CDK2 was performed. However, anti-CDK4 antibody (rabbit polyclonal IgG, Upstate Biotechnology Inc.) was used instead of anti-CDK2 antibody.

As a result, the compound HNP-98701 of the present invention had no effect on CDK4 of L929 and 293 cells (Table 11). Therefore, the apoptosis-type cell death in L929 and 293 cells by the compound HNP-98701 of the present invention is not related with CDK4 kinase activity inhibition during G1 phase.

TABLE 11
Determination of Reaction Inhibition Activity of
Compound HNP-98701 against CDK4 Kinase
of L929 L929 Cell Line

| | Sample Treatment | | |
|---|---|---|---|
| Cell Line | Concentration (μM) | Band Absorbance | Absorbance (%) |
| L929 | — | 0.754 | 100 |
| L929 | 100 | 0.692 | 91.5 |

<4-4> Determination of Cdc2 Kinase Activity

We also determined Cdc2 kinase activity of G2-M phase in order to know if HNP-98701 participates in G2-M phase and causes cell death.

The same procedure as that of <4-1> for CDK2 was performed. However, anti-Cdc2 antibody (mouse monoclonal IgG, Santa Cruz Biotechnology Inc.) was used instead of anti-CDK2 antibody.

As a result, the compound HNP-98701 of the present invention had no effect on Cdc2 kinase activity of 293 cell. Therefore, the apoptosis-type cell death in 293 cell by the compound HNP-98701 of the present invention is not related with Cdc2 kinase activity inhibition during G2-M phase.

<4-5> Determination of E2F-1 Protein Content Change

From Experimental Examples <4-1>-<4-4>, it was identified that the compound HNP-98701 of the present invention participates in G1-S phase of cell cycle and induces cell death. Based on this fact, we examined change of E2F-1 protein in order to know if HNP-98701 also participates in G1/S phase metastasis during cell cycle.

To be specific, 6×10⁵ cells/4 ml (in 60-mm dish) of liver cancer and prostate cancer cell lines were treated with HNP-98701 with different concentration and time. After culturing them overnight, the cells were collected and washed with PBS for two times. After dissolving the cells by adding 50 μl (/dish) of RIPA buffer solution, it was centrifuged (13,000 rpm, 10 min) to remove cell debris. Then, protein was quantized. After electrophoresing (80V stacking-120V running) the protein in SDS-PAGE, the protein on the gel phase was transferred to PVDF membrane (Millipore) using electric blotter (50V, 1.5 h-2 h), and then Western blot analysis was performed.

During Western blot analysis, the membrane was immersed in TBST solution containing 5% skim milk in order to inhibit non-specific reaction of antibody. While waving the solution for 1 hr at room temperature, the blank space where protein was not transferred was filled with skim milk. After adding E2F-1 antibody diluted to 1 μg/ml (in 2.5% skim-milk in TBST) to the membrane, primary antibody binding reaction was performed by waving for 1 hr at room temperature. Then, it was washed with TBST buffer solution for three times to remove primary antibody that did not bind to the membrane. After adding secondary antibody diluted to 1:10,000 to the membrane, secondary antibody binding reaction was performed by waving for 1 hr at room temperature. Then, it was washed with TBST buffer solution for three times to remove non-specifically bound secondary antibody. Then, it was exposed to X-ray film in order to determine band content.

As a result, cancer cell line (PC-3) treated with 1 μg/ml of the compound HNP-98701 of the present invention liver cancer cell line (SK-Hep-1) and prostate for 48 hr showed reduced E2F-1 protein content. This implies that apoptosis-type cell death caused by HNP-98701 in the cells are induced by inhibition of G1/S phase metastasis.

### EXPERIMENTAL EXAMPLE 5

### Effect of HNP-98701 on Kinase Related with Stress Activated Protein

In order to know the signal transfer path of the compound HNP-98701 of the present invention, kinase reaction activity related with stress activated protein was examined. For components related with stress activated protein, there are kinases like SAPK, p38 and MEKK. These induce apoptosis of cell through kinase activation if stress is transferred from outside. Therefore, from examination of kinase activity of SAPK, p38 and MEKK treated with the compound HNP-98701 of the present invention, one can know in which signal transfer path related with stress activated protein the compound HNP-98701 participates to induce apoptosis-type cell death.

<5-1> Determination of SAPK Kinase Reaction Activity

In order to know if apoptosis caused by the compound HNP-98701 of the present invention affects is related with SAPK path, we examined effect of HNP-98701 on SAPK kinase activity.

To be specific, various cell lines (L929, SK-Hep-1 and PC3 cells) were cultured on 100-mm tissue culture dish to 70-80%. Then, 1 μg/ml of HNP-98701 was treated by 5 ml for 30 min. Then, it was washed with PBS buffer solution for two times. The following procedure was the same as that of Experimental Example 2. However, anti-JNK antibody was used in place of anti-CDK2 antibody, and GST-Jun substrate was used instead of histone-H1. The cell line exposed by UV for 1 min and cultured for 1 hr was used as positive control group.

As a result, while SAPK kinase activity of L929 cell increased among the cell lines treated with the compound HNP-98701 of the present invention (Table 12), there was no change in SAPK kinase activity of PC3 and SK-Hep-1 cells. This result implies that while HNP-98701 may participate in SAPK path of L929 cell to induce apoptosis, apoptosis inducement in PC3 and SK-Hep-1 cells is unrelated with SAPK path. Therefore, the cell death mechanism in PC3 and SK-Hep-1 cells is not induced by promotion of SAPK kinase activation.

TABLE 12
Effect of HNP-98701 on SAPK Kinase Activity of L929 Cell Line

| | Treatment | | | |
|---|---|---|---|---|
| | Concentration | Treatment | Band | Absorbance |
| Cell Line | (μg/ml) | Time (min) | Absorbance | (%) |
| L929 | — | — | 0.013 | 1.3 |
| L929 | 1 | 30 | 0.445 | 43.6 |
| L929 | 1 | 60 | 0.360 | 35.2 |
| L929 | 1 | 180 | 0.030 | 2.9 |
| L929 | UV | 1 | 1.021 | 100 |

<5-2> Determination of MEKK Kinase Reaction Activity

In order to know if apoptosis caused by the compound HNP-98701 of the present invention affects is related with MEKK path, we examined effect of HNP-98701 on MEKK kinase activity.

To be specific, the same procedure as that of Experimental Example 2 was used. However, anti-MEKK antibody was used instead of anti-CDK2 antibody, and GST-SEK1 substrate was used in place of histone-H1. The cell line exposed by UV for 1 min and cultured for 1 hr was used as positive control group. Also, 0.5% of FBS was added to the culture medium and cultured for 18 hr to obtain serum-starved cell before treating with HNP-98701.

As a result, while the compound HNP-98701 of the present invention increased MEKK kinase activity of L929 by about 20%, there was no effect on PC-3 and 293 cells (Table 13). This result implies that while HNP-98701 may participate in path of L929 cell to induce apoptosis, apoptosis inducement in 293 and PC3 cell lines is related with MEKK path.

TABLE 13
Effect of HNP-98701 on MEKK kinase activity of L929, 293 and
PC-3 Cell Lines

| | Treatment | | | |
|---|---|---|---|---|
| | Concentration | Treatment | Band | Absorbance |
| Cell Line | (μg/ml) | Time (min) | Absorbance | (%) |
| L929 | — | — | 1.308 | 100.0 |
| L929 | 1 | 30 | 1.595 | 121.9 |
| L929 | UV | 1 | 1.649 | 126.1 |
| 293 | — | — | 0.270 | 100.0 |
| 293 | 1 | 30 | 0.271 | 100.4 |
| 293 | UV | 1 | 0.268 | 99.3 |
| PC-3 | — | — | 0.467 | 100.0 |
| PC-3 | 1 | 30 | 0.459 | 98.3 |
| PC-3 | UV | 1 | 0.533 | 113.5 |

### EXPERIMENTAL EXAMPLE 6

### Examination of Binding of HNP-98701 to Epidermal Growth Factor Receptor

If the compound HNP-98701 of the present invention is to induce apoptosis-type cell death, it should come into the cell or bind with cell receptor in order to transfer signal. We examined binding of HNP-98701 and epidermal growth factor receptor (hereunder referred to as “EGFR”) in order to know if this mechanism is intermediated by EGFR, which is a kind of protein kinase C (PKC). If the compound HNP-98701 of the present invention transfers signal via EGFR, autophosphorylation does not occur due to the binding of HNP-98701 and EGFR. Therefore, intermediation by EGFR can be identified by examining this.

To be specific, A431 cell, which is cell line for producing EGFR, was cultured on 100-mm tissue culture dish. Then, it was washed with cooled PBS buffer solution for two times. After adding a suitable amount of homogenizer buffer solution (50 mM tris-HCl, pH 7.4, 1 mM EDTA, 250 mM sucrose, 0.5 mM PMSF, 0.5 ug/ml leupeptin) to the cell, it was homogenized with glass homogenizer and centrifuged to remove cell debris. The obtained supernant was ultra-centrifuged to separate membrane fraction in precipitate form. Then, it was dissolved in buffer solution (homogenizer buffer+1% triton X-100) and ultra-centrifuged again to separate dissolved membrane fraction. After mixing this membrane fraction with a suitable amount of 20 mM HEPES, 10 mM Na₃VO₄ and HNP-98701 with 10 μM or 100 μM of final concentration. Then, EGFR was added so that the final concentration is to be 500 nM. EGFR was activated by letting this solution alone for 15 min at 30° C. After adding 300 mM MgCl₂, 300 μM ATP and r³²-ATP to the solution, reaction was performed for 5 min in ice. Then, the reaction was terminated by adding stop buffer and heating. After electrophoresing the reaction solution in 12% SDS-PAGE gel, it was exposed to X-ray film.

As a result, there was no change in autophosphorylation of EGFR by HNP-98701 treatment. Therefore, signal transfer of the compound HNP-98701 of the present invention is not mediated by EGFR, and it cause no effect on protein kinase C activity (Table 14).

TABLE 14
Effect of HNP-98701 on EFGR Autophosphorylation
of A431 Cell Line

| | Treatment | | | |
|---|---|---|---|---|
| | Concentration | Treatment | Band | Absorbance |
| Cell Line | (μM) | Time (min) | Absorbance | (%) |
| A431 | — | — | 3.832 | 100.0 |
| A431 | inhibitor | 20 | 0.534 | 13.9 |
| A431 | 10 | 20 | 3.876 | 101.1 |
| A431 | 100 | 20 | 4.141 | 108.3 |

### EXPERIMENTAL EXAMPLE 7

### Detection of Nitrogen Oxide Generation

Generation of nitric oxide, which is a toxic byproduct during apoptosis-typecell death, was examined.

To be specific, cell toxicity experiment was performed by the same procedure as that of Experimental Example 1. The cell line treated with HNP-98701 was cultured for only 48 hr. In order to detect nitric oxide, 50 μl of the culture medium was mixed with 50 μg of sulfanilamide solution and reacted for 5-10 min at room temperature in dark room. Then, 50 μl of NED solution was added and reaction was performed. Absorbance was measured at 520-550 nm. NO amount in the culture medium was determined as can be seen in Table 15. The remaining cell after NO detection was identified as dead by measuring existence rate and necrosis rate through MTT analysis and LDH analysis.

As a result, the compound HNP-98701 of the present invention was identified to not produce NO byproduct (Table 16). Therefore, the cell toxicity inducing mechanism of the compound HNP-98701 of the present invention is irrelevant of NO synthase.

TABLE 15
Absorbance according to NO Concentration

| NO Concentration (μM) | Band Absorption |
|---|---|
| 50 | 0.343 |
| 25 | 0.197 |
| 12.6 | 0.131 |
| 6.25 | 0.102 |
| 3.13 | 0.079 |

TABLE 16
Effect of HNP-98701 on Activity of NO Synthase

| | | Treatment | | | |
|---|---|---|---|---|---|
| | | Concen- | NO | MTT | LDH |
| Items | Sample | tration | Analysis | Analysis | Analysis |
| Blank | Medium | — | 0.061 | | |
| | Treatment | | | | |
| Control | Cell + | — | 0.055 | 100% | 0% |
| Group | Medium | | | | |
| Sample | HNP-98701 | 1 μg/ml | 0.056 | 13.97% | 78.83% |
| Treatment | | | | | |
| Sample | HNP-98701 | 0.1 μg/ml | 0.057 | 0.40% | 84.48% |
| Treatment | | | | | |
| Triton | Triton X-100 | 1% | | | 100% |
| Treatment | | | | | |

### EXPERIMENTAL EXAMPLE 8

### Determination of Adhesion Inhibition Activity of HNP-98701 Against Cancer Cell Metastasis

In order to identify effect of HNP-98701 on cancer cell metastasis, we determined inhibition activity of HNP-98701 against adhesion to blood vessel wall of cancer cell.

In general, cancer cell metastasis occurs because cancer cell penetrates into blood vessel or transfers to other organ or tissue. The most important process in cancer cell metastasis is its adhesion to blood vessel wall of other organ or tissue. Therefore, if the compound HNP-98701 of the present invention affects adhesion of cancer cell to blood vessel wall, one can say that it also affects cancer cell metastasis. In order to identify this, we examined adhesion inhibition activity of HNP-98701 against blood vessel wall matrix components related with adhesion to cancer cell, such as matri-gel, laminin, collagen and gelatin.

After treating HNP-98701 on tissue culture plate coated with the matrices, cancer cell was attached thereupon. To be specific, 50 μl/ml of matri-gel, 2 μg/ml of laminin, 2 μg/ml of collagen and 2 μg/ml of gelatin were coated on the surface of 96-well microplate (Corning Co.) per well. After solidifying for 2 hr, HNP-98701 was added to each well and treated for 1 hr. HNP-98701 was dissolved in DMSO, so that the final concentration becomes 10 μg/ml, 1 μg/ml and 0.1 μg/ml, respectively. Cisplatin with the final concentrations being 50 μg/ml, 5 μg/ml and 0.5 μg/ml and adriamycin with the final concentrations being 10 μg/ml, 1 μg/ml and 0.1 μg/ml were used as control groups. After treating the substances for 1 hr, cancer cell was added to each cell, and it was cultured for 3 hr. For the cancer cell, liver cancer cell line (SK-Hep-1), malignant melanosis cell line (SK-MEL-28), throat cancer cell line (Hep-2) and normal cell line (NIH-3T3) were used so that the final concentration to be 1×10⁶ cells/ml. After culturing these cell lines for 3 hr, they were centrifuged. After collecting biomass only, it was washed with distilled water or PBS buffer solution for three times. After dying the cell with Diff-Quick solution (Fisher Scientific), it was treated with 1% SDS/0.5N NaOH to dissolve attached cells. Then, absorbance was measured at 560-405 nm with automatic Eliza detector (Molecular Divices Corp., USA). Cell adhesion rate (%) was determined by Equation 1 as in Experimental Example 1. Each experiment was performed for three times repeatedly.

As a result, the compound HNP-98701 of the present invention showed no inhibition effect against adhesion activity of cancer cell to various matrices, as for cisplatin and adriamycin (Table 17, Table 18, Table 19 and Table 20). Therefore, the compound HNP-98701 of the present invention has no inhibition activity against adhesion of cancer cell to blood vessel.

TABLE 17
Determination of Inhibition Activity of HNP-98701 against
Adhesion of Cancer Cell to Matri-Gel

| | Treatment | | |
|---|---|---|---|
| Sample | Concentration (μg/ml) | SK-Hep-1 (%) | NIH-3T3 (%) |
| HNP-98701 | 10 | 95.21 | 108.48 |
| | 1 | 102.04 | 115.29 |
| | 0.1 | 103.23 | 112.31 |
| Cisplatin | 50 | 108.26 | 101.06 |
| | 5 | 105.27 | 102.40 |
| | 0.5 | 96.41 | 105.67 |
| Adriamycin | 10 | 88.74 | 104.23 |
| | 1 | 95.93 | 108.65 |
| | 0.1 | 99.04 | 115.00 |
| Control Group | — | 100.00 | 100.00 |

TABLE 18
Determination of Inhibition Activity of HNP-98701 against
Adhesion of Cancer Cell to Laminin

| | Treatment | SK- | | | |
|---|---|---|---|---|---|
| | Concentration | Hep-1 | NIH-3T3 | SK-MEL- | Hep-2 |
| Sample | (μg/ml) | (%) | (%) | 28 (%) | (%) |
| HINP-98701 | 10 | 92.68 | 103.17 | 107.88 | 107.26 |
| | 1 | 101.91 | 101.91 | 107.14 | 108.64 |
| | 0.1 | 99.58 | 100.00 | 105.99 | 109.39 |
| Cisplatin | 50 | 88.74 | 92.45 | 101.16 | 95.87 |
| | 5 | 93.53 | 94.94 | 105.36 | 101.25 |
| | 0.5 | 95.65 | 93.86 | 110.19 | 102.63 |
| Adriamycin | 10 | 95.86 | 91.63 | 113.03 | 98.75 |
| | 1 | 96.92 | 103.07 | 109.66 | 105.13 |
| | 0.1 | 96.18 | 107.38 | 108.40 | 106.76 |
| Control | — | 100.00 | 100.00 | 100.00 | 100.00 |
| Group | | | | | |

TABLE 19
Determination of Inhibition Activity of HNP-98701 against
Adhesion of Cancer Cell to Collagen

| | Treatment | | | | |
|---|---|---|---|---|---|
| | Concen- | | | | |
| | tration | SK-Hep- | NIH-3T3 | SK-MEL- | Hep-2 |
| Sample | (μg/ml) | 1 (%) | (%) | 28 (%) | (%) |
| HNP-98701 | 10 | 91.21 | 115.06 | 90.75 | 97.74 |
| | 1 | 93.55 | 133.30 | 96.15 | 101.13 |
| | 0.1 | 101.76 | 123.01 | 98.79 | 102.51 |
| Cisplatin | 50 | 95.90 | 98.94 | 117.62 | 82.83 |
| | 5 | 104.57 | 108.91 | 106.83 | 89.35 |
| | 0.5 | 104.22 | 112.51 | 98.46 | 92.23 |
| Adriamycin | 10 | 103.28 | 111.13 | 94.71 | 88.85 |
| | 1 | 97.89 | 109.97 | 94.49 | 93.11 |
| | 0.1 | 98.83 | 111.98 | 94.27 | 91.10 |
| Control Group | — | 100.00 | 100.00 | 100.00 | 100.00 |

TABLE 20
Determination of Inhibition Activity of HNP-98701 against
Adhesion of Cancer Cell to Gelatin

| | Treatment | | | | |
|---|---|---|---|---|---|
| | Concen- | | | | |
| | tration | SK-Hep- | NIH-3T3 | SK-MEL- | Hep-2 |
| Sample | (μg/ml) | 1 (%) | (%) | 28 (%) | (%) |
| HNP-98701 | 10 | 37.99 | 107.05 | 92.37 | 83.47 |
| | 1 | 77.99 | 115.62 | 94.22 | 107.07 |
| | 0.1 | 77.51 | 113.81 | 96.18 | 111.33 |
| Cisplatin | 50 | 61.25 | 100.10 | 86.40 | 91.07 |
| | 5 | 74.84 | 110.48 | 91.13 | 100.13 |
| | 0.5 | 79.16 | 111.43 | 92.48 | 97.20 |
| Adriamycin | 10 | 33.29 | 105.81 | 97.21 | 97.73 |
| | 1 | 76.37 | 108.86 | 97.42 | 98.27 |
| | 0.1 | 85.77 | 108.67 | 96.39 | 104.27 |
| Control Group | — | 100.00 | 100.00 | 100.00 | 100.00 |

### EXPERIMENTAL EXAMPLE 9

### Determination of Angiogenesis and Metastasis Inhibition Effect of HNP-98701

In order to identify inhibition effect of HNP-98701 other than blood vessel wall adhesion inhibition, we determined inhibition effect against MMP-2 (matrix metalloproteinase-2), which is an important enzyme in angiogenesis and metastasis of cancer cell.

Angiogenesis refers to generation of new blood vessel in existing capillary vessel. For angiogenesis to occur, basement membrane of the existing blood vessel should be broken down first. MMP-2 is the enzyme that breaks down the basement membrane. Also, cancer cell metastasis is accompanied by growth of cancer cell, because cancer cell inside epidermal tissue draws in blood vessel transferred to corium. In doing so, for the cancer cell in epidermal tissue to be able to move to corium, basement membrane between the epidermal tissue and the cancer cell should be broken down. MMP-2 also participates in this process.

In determination of MMP-2 inhibition activity, 7 peptides comprising mainly tryptophane are usually used. If these peptides are treated with DNP, generation of fluorescence is inhibited. If the fluorescence-inhibited peptide is treated with MMP-2, DNP is removed and fluorescence is recovered. Activity of MMP-2 is determined by this fluorescence. To be specific, baculovirus expression system was used to obtain proMMP-2. Then, it was treated with 1 mM p-(aminophenyl)mercuric acetate at 37° C. to obtain activated MMP-2. The peptide used as reaction substrate comprises Pro-Leu-Met-Trp-Ser-Arg, and DNP-Pro-Leu-Met-Trp-Ser-Arg is formed if it is treated with fluorescence inhibition material. Buffer solution comprising 50 mM tricine, 0.2M NaCl and 10 mM CaCl₂ with pH adjusted to 7.5 was used for search reaction. The search reaction was performed at 23° C. After adding MMP-2 to buffer solution containing the peptide and HNP-98701, it was stabilized by storing it at room temperature. Then, fluorescence change was determined with a fluorometer (Perkin-Elmer Model LS 50B fluorometer).

As a result, in spite of treatment with the compound HNP-98701 of the present invention, fluorescence was induced by removal of DNP, the fluorescence inhibition material, from the peptide by the enzymeMMP-2. Therefore, the compound HNP-98701 of the present invention did not inhibit activity of MMP-2, which is an important enzyme in angiogenesis and cancer cell metastasis.

### EXPERIMENTAL EXAMPLE 10

### Animal Toxicity Experiment

In order to find out toxicity of the compound HNP-98701 of the present invention, we injected HNP-98701 of various concentrations to laboratory animals intravenously. Then, 50% lethal dose (LD₅₀), behavioral characteristics and weight change of the laboratory animals were observed for 2 weeks. For laboratory animals, five 6-week-old ICR mice weighing 30.0-30.8 g were used for each group. They were starved for 24 hr before intravenous injection. HNP-98701 was dissolved in DMSO, which has low cell toxicity. The administration does was determined, based on LD₅₀ of in vitro experiment, to be 10,000 times (0.5 mg/head), 5,000 times (0.25 mg/head) and 2,500 times (0.125 mg/head). For control group, DMSO without containing HNP-98701 was administered. After injecting each concentration of HNP-98701 into tail vein of mouse, death rate, anomaly like behavioral change, alopecia and flare and weight change were observed for 15 days every 2-3 days.

As a result, while the mice administered 0.5 mg/head and 0.25 mg/head of HNP-98701 all died, the ones administered 0.125 mg/head of HNP-98701 all survived. Also, the mice alive showed the same behavioral pattern as those of control group, to which only DMSO was administered. And, there was no anomaly like special behavioral characteristics, alopecia or flare. Therefore, 50% lethal dose (LD₅₀) of the compound HNP-98701 of the present invention was identified to be in the concentration range of 0.125 mg/head-0.25 mg/head (4.12 mg/kg-8.25 mg/kg) (Table 21). This almost coincides with the 50% lethal dose of manassantin A, which was reported to be LD₅₀=5.2 mg/kg by Rao et al. Also, the therapeutic index of HNP-98701 was calculated by Equation 3 from 50% effective concentration against prostate cancer cell line and toxicity against laboratory animal in in-vitro experiment. As a result, the therapeutic index (T.I.) of the compound HNP-98701 of the present invention was larger than 2,500.

<Equation 3>

Therapeutic Index=(Lethal Concentration 50% Kill against Laboratory Animal)/(Effective Concentration 50% against Cancer Cell Line)

Comparing this result with that of manassantin A intraperitoneally administered to mouse, which showed spontaneous movement 50% reduction concentration (IC₅₀) to be 0.21±0.02 mg/kg, reported by Rao et al., HNP-98701 of the present invention shows anticancer activity (IC₅₀) at a much lower concentration of about 1/100. Therefore, the compound HNP-98701 of the present invention is very selective in that it has very strong cell toxicity against cancer cell but very low toxicity against normal cell. Therefore, it can be very useful in development of superior carcinostatis substance having strong anticancer activity and few adverse effects.

TABLE 21
Animal Toxicity Experiment Result for HNP-98701

| Treatment | | | | | | | | | | |
|---|---|---|---|---|---|---|---|---|---|---|
| Concentration | Weight Change of Mice before & after Sample Treatment | | | | | | | | | |
| (mg/head) | −1 | 0 | 1 | 2 | 3 | 5 | 7 | 9 | 12 | 15 |
| 0.125 | 30.5 | 27.1 | 31.2 | 32.9 | 33.7 | 34 1 | 34.7 | 35.5 | 35.6 | 37.2 |
| 0.25 | 30.4 | 27.2 | — | — | — | — | — | — | — | — |
| 0.5 | 30.5 | 27.0 | — | — | — | — | — | — | — | — |
| Control Group | 30.6 | 27.3 | 31.5 | 32.5 | 34.1 | 35.1 | 35.9 | 36.6 | 38.5 | 38.8 |

The present invention provides use of the novel compounds HNP-98701A and HNP-98701B and known compound HNP-98701C, or mixture thereof (HNP-98701) extracted from saururus as carcinostatis substance, preparation method thereof and carcinostatis pharmaceutical composition containing them as effective constituent. The carcinostatis substances HNP-98701A, HNP-98701B, HNP-98701C, mixture thereof (HNP-98701) and derivatives thereof show anticancer activity against cancer cell lines, including liver cancer, prostate cancer, breast cancer, throat cancer, glioblastoma and malignant melanosis much stronger than that of the conventional carcinostatis substance like cisplatin or adriamycin. And it shows little or no adverse effect against normal cell. Therefore, development of very superior and selective carcinostatis substance is expected.

Also, the carcinostatis substance containing HNP-98701A, HNP-98701B, HNP-98701C, mixture thereof (HNP-98701) or derivatives thereof as effective constituent can be developed in the form of oral-administration drug. Therefore, it can be administered easily and used together with the conventional carcinostatis substances or therapies. Besides, it can be used as effective constituent of carcinostatis functional food or food additive, carcinostatis functional drink or drink additive, carcinostatis cosmetic additive, carcinostatis soap additive and carcinostatis shampoo additive.

In addition, because saururus can be grown easily, it can contribute to income growth of rural communities. And, because saururus or its root can be used as live herb or dried herb and even the residue can be utilized, saururus contributes to recycling of resources.

And, because hydroxyl group (—OH) of the carcinostatis substances HNP-98701A and HNP-98701C is highly reactive, these substances can be used as lead compounds for synthesis of derivatives, search of activity and development of new drugs.

What is claimed is:

1. A novel compound epi-manassantin A expressed by Formula 1, its derivative or pharmaceutically available salt thereof, which has anticancer activity: 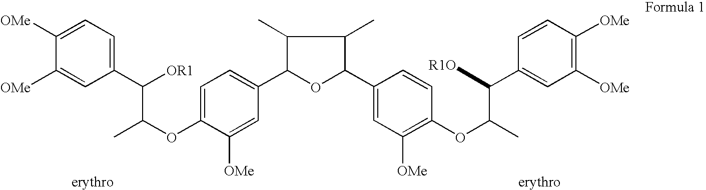
In Formula 1, R1 is selected from methyl, acetyl, alkyl, alkene, alkyne, amine, amide, cyano, thiocyano, aldehyde or halogen atom, and may be identical or different.

2. A compound, its derivative or pharmaceutically available salt thereof according to claim 1, wherein R1 is selected from H, CH₃ or COCH₃.

3. A novel compound expressed by Formula 2, its derivative or pharmaceutically available salt thereof, which has anticancer activity: 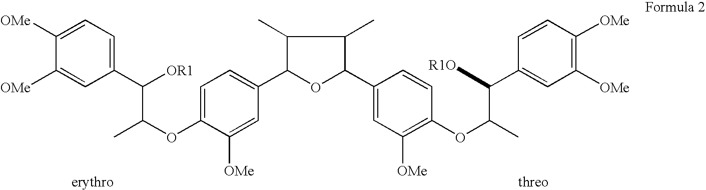
In Formula 2, R1 is selected from methyl, acetyl, alkyl, alkene, alkyne, amine, amide, cyano, thiocyano, aldehyde or halogen atom, and may be identical or different.

4. A compound, its derivative or pharmaceutically available salt thereof according to claim 3, wherein R1 is selected from H, CH₃ or COCH₃.

5. A compound manassantin A expressed by Formula 3, its derivative or pharmaceutically available salt thereof, which has anticancer activity: 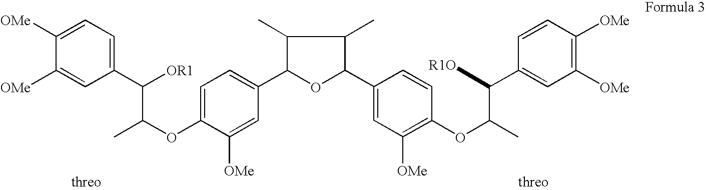
In Formula 3, R1 is selected from methyl, acetyl, alkyl, alkene, alkyne, amine, amide, cyano, thiocyano, aldehyde or halogen atom, and may be identical or different.

6. A compound, its derivative or pharmaceutically available salt thereof according to claim 5, wherein R1 is selected from H, CH₃ or COCH₃.

7. A compound expressed by Formula 1, Formula 2 or Formula 3, or decomposed substance thereof, which has anticancer activity and has been separated from saururus.

8. A preparation method of a compound expressed by Formula 1, Formula 2 or Formula 3, or derivative thereof, which has anticancer activity and has been separated from saururus, which comprises:
1) A step of immersing saururus in low alcohol and repeatedly extracting it;
2) A step of extracting the low alcohol extract with various organic solvents;
3) A step of separating the various organic solvent extracted fractions with chromatography to obtain carcinostatis substances; and
4) A step of purifying each separated carcinostatis substance through solvent precipitation.

9. A preparation method according to claim 8, wherein the saururus in step 1) includes residue remaining after using it as live herb, dried herb, stem top, root and tea.

10. A preparation method according to claim 8, wherein the low alcohol in step 1) is methanol; and the various organic solvents in step 2) are selected from hexane, chloroform, ethyl acetate or butanol.

11. An extract derived from saururus having anticancer activity, which is prepared in each step of the preparation method according to claim 8.

12. A carcinostatis pharmaceutical composition containing a compound expressed by Formula 1, Formula 2 or Formula 3, derivative thereof or pharmaceutically available salt thereof as effective constituent, which has anticancer activity and has been separated from saururus.

13. A carcinostatis pharmaceutical composition according to claim 12, wherein the compound expressed by Formula 1, Formula 2 or Formula 3 is included in the range of 0.1-3.0 mg/kg.

14. A carcinostatis pharmaceutical composition according to claim 12, which is effective in liver cancer, prostate cancer, breast cancer, mammary gland cancer, uterus epidermal cancer, glioma, neuroblastoma, glioblastoma, stomach cancer, throat epidermal cancer, fibrous carcinoma or malignant melanosis, and preferably effective in liver cancer, prostate cancer, mammary gland cancer, glioblastoma or malignant melanosis.

15. A functional food, food additive, functional drink or drink additive containing a compound expressed by Formula 1, Formula 2 or Formula 3 or derivative thereof as effective constituent, which has anticancer activity.

16. A skin-care product additive containing a compound expressed by Formula 1, Formula 2 or Formula 3 or derivative thereof as effective constituent, which has anticancer activity.

17. A skin-care product additive according to claim 16, which is cosmetic additive, soap additive or shampoo additive.

18. A carcinostatis substance composition containing more than two compounds selected from the compounds expressed by Formula 1, Formula 2 or Formula 3.

19. A carcinostatis substance composition which contains saururus extract.
